(19) United States
(12) Patent Application Publication
(10) Pub. No.: US 2026/0004930 A1
(43) Pub. Date: Jan. 1, 2026

(54) AUTOMATED CAREGIVER SYSTEMS AND METHODS

(71) Applicant: Voxela, Inc, Fremont, CA (US)

(72) Inventors: Goutham Kamath, Fremont, CA (US); Yuta Endo, Santa Clara, CA (US); Yusuke Yoshida, Tokyo (JP)

(21) Appl. No.: 19/251,294

(22) Filed: Jun. 26, 2025

Related U.S. Application Data
(60) Provisional application No. 63/665,465, filed on Jun. 28, 2024.

(51) Int. Cl. G16H 40/67; G06V 20/40; G06V 20/52; H04N 7/18

(52) U.S. Cl. CPC G16H 40/67; G06V 20/44; G06V 20/52; H04N 7/183

(57) ABSTRACT

Systems and methods for automated caregiving are disclosed. One aspect includes a sensing system configured to monitor an individual, including at least one camera and at least one microphone. The at least one camera may be configured to capture any combination of video and images of an individual being monitored. The microphone may be configured to capture audio signals generated by the individual. An edge computing system connected to the sensing system may be configured to receive one or more sensed signals associated with the sensing system monitoring the individual. The sensed signals may include the video or images and the audio signals. A remote computing system connected to the edge computing system via a network may be configured to receive the sensed signals from the edge computing system, process the sensed signals using an artificial intelligence (AI)-based system, and provide assistance to the individual being monitored.

### BACKGROUND

This application claims the priority benefit of provisional patent application No. 63/665,465 titled “AI Caregiver System with Video & Audio Analysis for Senior Care Facilities & Hospitals” filed on Jun. 28, 2024, the disclosure of which is incorporated by reference herein in its entirety.

### TECHNICAL FIELD

The systems and methods described herein relate to the use of artificial intelligence (AI) systems and algorithms to provide an enhanced caregiving capability for senior care facilities and hospitals.

### BACKGROUND ART

The current state of senior care faces challenges due to the growing population of older adults, rising healthcare costs, and a shortage of qualified caregivers. Existing technologies, such as medical alert systems, medication dispensers, and fall prevention devices, offer support for seniors' safety and independence. However, these technologies often lack real-time monitoring and assistance, leaving seniors vulnerable in emergencies.

The increasing number of older adults strains the current senior care system. The shortage of qualified caregivers exacerbates the situation, leading to higher healthcare costs. In addition, continuous human-based monitoring of seniors living alone or with health conditions can increase the workload and strain on existing caregivers.

While existing senior care technologies contribute to helping improve safety and independence of seniors, these technologies lack real-time monitoring and assistance. This gap poses risks for seniors living alone or with health conditions requiring close monitoring.

Senior care facilities require improved monitoring and assistance to ensure residents' safety and well-being. This includes tracking residents' movements, monitoring vital signs, and providing emergency assistance in an automated manner and with minimal human supervision or intervention.

Existing monitoring and assistance solutions for senior care facilities often fall short of providing a solution that includes comprehensive autonomous monitoring. In addition, these solutions may be expensive, challenging to use, and lack real-time capabilities.

Enhanced monitoring and assistance are, therefore, necessary for senior care facilities to ensure residents' safety and well-being. Real-time monitoring, movement tracking, vital sign monitoring, and emergency assistance are crucial components of an autonomous monitoring system.

Many senior care facilities struggle to meet their residents' care needs due to shortcomings in existing monitoring and assistance solutions. Any available contemporary solutions often come with high costs, usability challenges, and a lack of real-time capabilities.

### SUMMARY

Aspects of the invention are directed to artificial intelligence-based systems and methods that enhance caregiver effectiveness for monitoring seniors in their respective senior care facilities. One aspect includes a sensing system configured to monitor an individual. The sensing system may include at least one camera and at least one microphone. The at least one camera may be configured to capture any combination of video and images of an individual being monitored. The least one microphone may be configured to capture audio signals generated by the individual. The system may also include at least one speaker to send one or more audio signals to the individual.

The system may also include an edge computing system connected to the sensing system and configured to receive one or more sensed signals associated with the sensing system monitoring the individual. In an aspect, the sensed signals include the video or images, and the audio signals.

One aspect includes a remote computing system connected to the edge computing system via a network. The remote computing system may be configured to receive the sensed signals from the edge computing system process the sensed signals using at least one artificial intelligence (AI)-based system, and provide assistance to the individual being monitored based on the processing.

Other aspects include methods that define one or more algorithms that can be implemented on the above system.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram of a computer architecture configured to implement an automated caregiver system.

FIG. 2 is a block diagram depicting a computer architecture interface.

FIG. 3 is a screenshot depicting an interaction between a human caregiver and an automated caregiver system.

FIGS. 4A and 4B are flow diagrams depicting a method for automated caregiving.

FIG. 5 is a block diagram of a computing system.

### DETAILED DESCRIPTION

In the following description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random-access memory (RAM) device, a read-only memory (ROM) device, an erasable programmable read-only memory (EPROM or Flash memory) device, a portable compact disc read-only memory (CDROM), an optical storage device, a magnetic storage device, and any other storage medium now known or hereafter discovered. Computer program code for carrying out operations of the present disclosure may be written in any combination of one or more programming languages. Such code may be compiled from source code to computer-readable assembly language or machine code suitable for the device or computer on which the code can be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“SaaS”), Platform as a Service (“PaaS”), and Infrastructure as a Service (“IaaS”)), and deployment models (e.g., private cloud, community cloud, public cloud, and hybrid cloud).

The flow diagrams and block diagrams in the attached figures illustrate the architecture, functionality, and operation of possible implementations of systems, methods, and computer program products according to various embodiments of the present disclosure. In this regard, each block in the flow diagrams or block diagrams may represent a module, segment, or portion of code, which includes one or more executable instructions for implementing the specified logical function(s). It is also noted that each block of the block diagrams and/or flow diagrams, and combinations of blocks in the block diagrams and/or flow diagrams, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flow diagram and/or block diagram block or blocks.

Aspects of the systems and methods described herein are related to an automated artificial intelligence (AI)-based system that enhances the efficiency of caregivers and improves patient safety. One aspect includes a sensing system that may further include a camera integrated with video, audio, and microphone capabilities for continuous monitoring in healthcare environments, such as monitoring a resident in their room. The system (also referred to herein as an “AI caregiver system” or an “AI caregiver”) may incorporate a locally-run AI model within the camera unit or some other edge computing device, complemented by a centralized AI processing system for advanced decision-making. This dual-model approach allows for 24/7 resident surveillance, anomaly detection in sounds and movements, and remote interaction by human caregivers through two-way audio.

In one aspect, the AI caregiver system enables human caregivers to ask any question related to the room and/or residents. The system responds with a textual answer and/or a link to a short video clip relevant to the question. This feature enhances caregiver assurance and understanding of the situation associated with the room and/or the residents.

In an embodiment, the AI caregiver system divides its functionality into three categories: Alerting, Monitoring, and Visual Question and Answer. Each category aims to improve caregiver efficiency and patient safety. The AI Caregiver system described herein is designed for use in various healthcare settings, including hospitals, senior care facilities, and skilled nursing facilities.

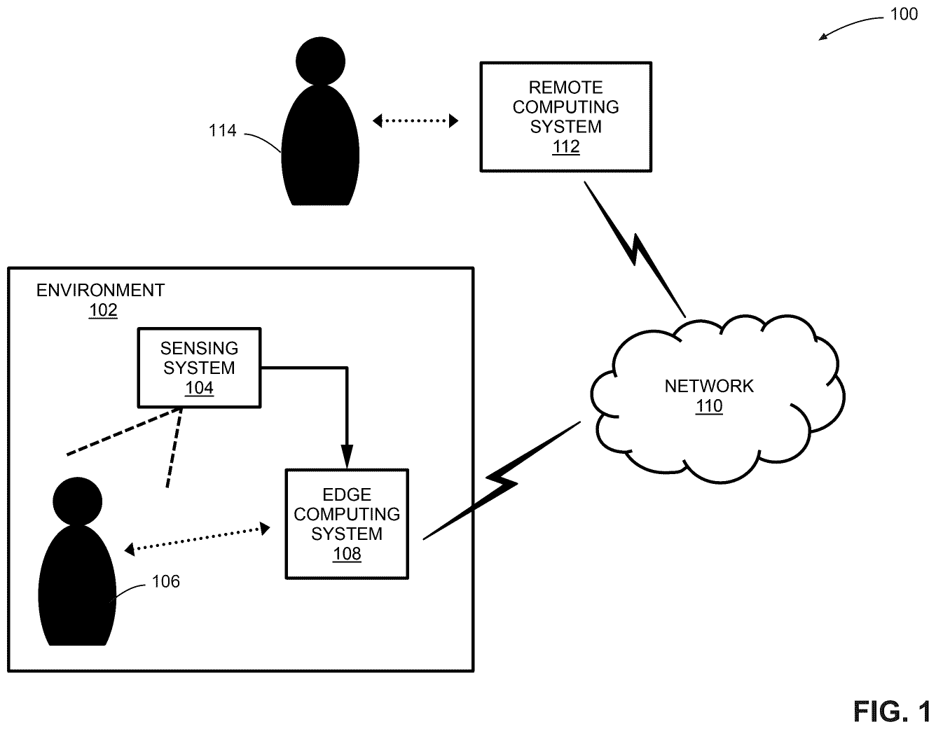

FIG. 1 is a block diagram of a computer architecture 100 configured to implement an automated caregiver system/AI caregiver system/AI caregiver. As depicted, computer system architecture 100 includes environment 102, network 110, and remote computing system 112. Environment 102 further includes sensing system 104 and edge computing system 108.

In an aspect, environment 102 may be a room or a living environment in a setting such as a senior living center/care facility, an assisted living center, a skilled nursing facility, a hospital, etc. Sensing system 104 may be configured to monitor daily activities of individual 106. Individual 106 may be a senior resident, a patient, or some other individual that needs monitoring.

In an aspect, sensing system 104 includes one or more sensors that are configured to monitor individual 106. The sensors included in sensing system 104 include, for example, one or more cameras configured to capture still images or video of individual 106. These still images or video may include images and/or video of individual 106 engaging in daily activities. Sensing suite 104 may also include one or more microphones configured to capture audio data associated with individual 106. The audio data may include sounds produced by individual 106 while engaging in daily activities. Other sensors that may be included in sensing suite 104 are depth sensors, radar sensors, infrared (IR) sensors, etc.

In an aspect, sensing suite 104 generates sensing data from the one or more included sensors. The sensing data may include audio/visual data, radar sensing data, depth sensing data, infrared data, etc. The sensing data may be received by edge computing system 108. Edge computing system 108 may be configured to receive and process a portion of the sensing data, or perform no processing on the sensing data. Edge computing system 108 may perform the processing using one or more AI-based systems.

Edge computing system 108 may be communicatively coupled with remote computing system 112 via network 110. Edge computing system 108 may transmit all or a portion of the sensing data received from sensing system 104 to remote computing system 112 via network 110. Edge computing system 108 may also transmit results from processing the portion of the data to remote computing system 112 via network 110. Network 110 may be a computer communication channel such as the Internet, an intranet, a local area network (LAN), or some other kind of computer communication channel.

In an aspect, remote computing system 112 uses one or more AI-based systems to process the sensing data received from edge computing system 108. Healthcare provider 114 may be presented with the processing results from edge computing system 108 and remote computing system 112 by remote computing system 112. Healthcare provider 114 may review the results and take any necessary action for the wellbeing of individual 106.

In an aspect, healthcare provider 114 and individual 106 may be able to interact with each other via a combination of remote computing system 112 and edge computing system 108, respectively. For example, a connection between remote computing system 112 and edge computing system 108 via network 110 may enable healthcare provider 114 to interact with individual 106 via audio conferencing/an audio call, or video conferencing/a video call, or some combination of an audio/video interaction.

In an aspect, edge computing system 108 is any of a laptop computer, a desktop computer, a mobile computing device (e.g., a tablet or a mobile phone), or some other computing device. Edge computing system 108 may also be implemented as a single-board computing system. In an aspect, remote computing system 112 is any of a server, a cloud computing system, and so on. As used herein, the term “computing system” generally refers to a device that includes at least one processor, a memory, and a network interface.

In one aspect, healthcare provider 114 may be able to interact, for example via text messaging, with an AI agent deployed on remote computing system 112. An interaction between healthcare provider 114 and the AI agent may allow the healthcare provider 114 to ask questions to the AI agent, such as questions about the well-being of individual 106. The AI agent may process the data received from edge computing system 108 and/or access data already processed by any combination of remote computing system 112 and edge computing system 108, to answer the questions posed to the AI agent by healthcare provider 114, while also providing supporting data such as video data, one or more images, and/or audio data associated with sensing system 104 monitoring individual 106. Such interactive sessions between healthcare provider 114 and the AI agent may allow healthcare provider 114 to make informed decisions regarding the well-being of individual 106.

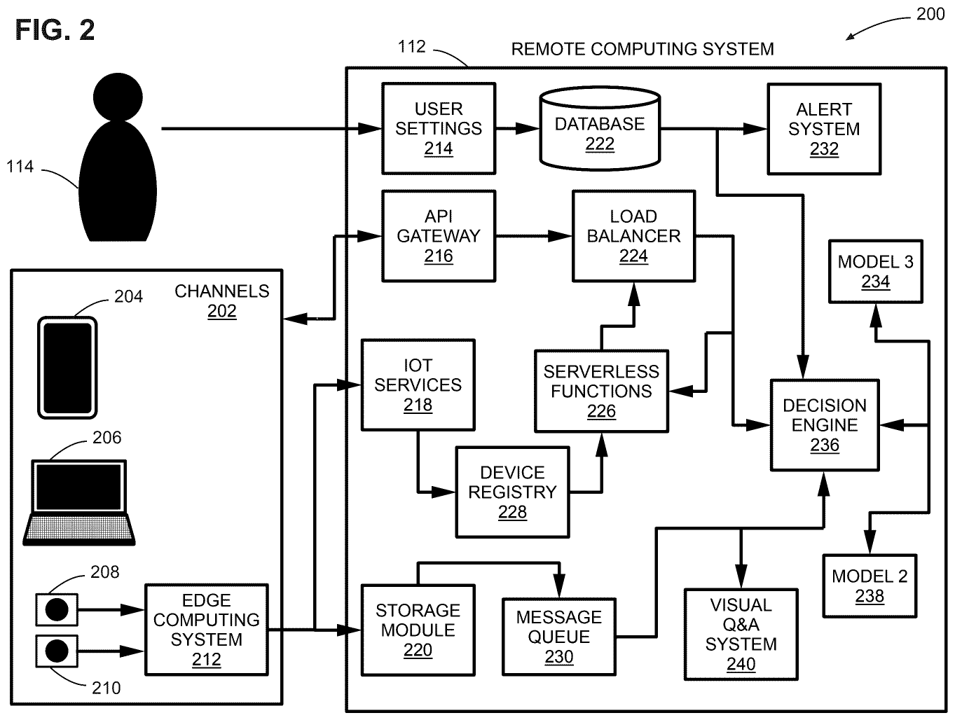

FIG. 2 is a block diagram depicting a computer architecture interface 200. As depicted, interface 200 is an interface between channels 202 and remote computing system 112. In an aspect, channels 202 includes components present in environment 102 (i.e., sensing system 104 and edge computing system 108). As shown in FIG. 2, channels 202 includes a mobile device 204, a laptop computer 206, cameras 208 and 210, and an edge computing system 212. In one aspect, cameras 208 and 210 are integrated into sensing system 104. Edge computing system 212 may be similar to edge computing system 108. In a particular embodiment, cameras 208 and 210 are integrated into edge computing system 212.

In an aspect, remote computing system 112 is deployed as a cloud server. Remote computing system 112 further includes user settings 214, API gateway 216, IoT services 218, storage module 220, database 222, load balancer 224, serverless functions 226, device registry 228, message queue 230, alert system 232, decision engine 236, visual Q&A system 240, model 3 234, and model 2 238.

User settings 214 may be associated with user account information and preferences for healthcare provider 114. These user settings may be stored on database 222. The database 222 may be a no sequential query language (SQL) database. Using a NoSQL database offers flexibility to store diverse and evolving data formats, such as video metadata, alerts, and caregiver notes, without requiring a fixed schema. If a new feature such as an AI generated report needs to be added, a new Key/Value pair may be added to the database 222 without requiring any changes to the existing table. Such an implementation allows real-time, scalable access to large volumes of sensor and event data across multiple residents and facilities. This further enhances system responsiveness and supports continuous learning models by enabling quick storage and retrieval of unstructured and semi-structured data.

In an aspect, the user account information stored to databases 222 may be used for user authentication. Once authenticated, remote computing system 112 may enable alert system 232, that displays one or more alerts to healthcare provider 114, regarding individual 106. Outputs from database 222 may also be used to inform decision engine 236, that healthcare provider 114 is logged in, and that any alerts associated with individual 106 (and any other individuals assigned to be monitored by healthcare provider 114) are to be provided to healthcare provider 114 via alert system 232.

In an aspect, API gateway 216 is configured to manage and route incoming API requests coming from a user mobile device or webUI to appropriate backend services securely, handling authentication, throttling, and logging. Load balancer 224 may be configured to distribute incoming network traffic across multiple servers to ensure high availability and reliability of services. In an aspect, IoT services 218 facilitates secure communication, monitoring, and management of connected internet-of-things (IOT) cameras (e.g., cameras 208 and 210) deployed in facilities (e.g., in environment 102), and helps manage remote over the air deployment, health check etc. Device registry 228 is configured to maintain metadata and status of all registered IoT cameras, enabling lifecycle management and identification.

In one aspect, serverless functions 226 executes backend logic in response to events without managing servers, enabling scalable and event-driven processing. Storage model 220 may be configured to define how video, sensor, and metadata are stored-supporting structured and unstructured formats with efficient retrieval and tagging. Message queue 230 buffers and manages asynchronous communication between components to ensure reliability and decoupled workflows. Visual Q&A System 240 may be configured to provide an interactive interface that allows users to query video or event data visually, supporting explainable AI outputs and caregiver insights.

### Hardware Components

### Camera Specifications:

In an aspect, cameras 208 and 210 may include an integrated central processing unit (CPU) and Deep Learning Processing Unit (DPU), custom-designed to manage AI models and data-intensive tasks. This integration enables real-time video analysis directly on the device (i.e., on edge computing device 212). In this case, edge computing system 212/108 is directly integrated into sensing system 104 (that includes cameras 208 and 210). Edge computing system 212 may transmit data to IoT services 218 and storage module 220. In an aspect, edge computing system 212 may include an AI model (model 1, not depicted in FIG. 2).

Equipped with high-definition video capture at 1080p resolution, the cameras 208 and 210 are enabled to adapt to both daytime and nighttime conditions through an infrared (IR) illumination feature. This ensures uninterrupted monitoring regardless of lighting variations.

For enhanced communication, the camera(s) 208 and 210 can support two-way audio communication, utilizing both a microphone and speaker. This facilitates interactive sessions between caregivers (e.g., healthcare provider 114) and residents (e.g., individual 106), enabling real-time conversations and interactions. These interactions may be enabled for individual 106 via any combination of mobile device 204 and laptop computer 206. For example, audio/video communication software on any combination of mobile device 204 and laptop computer 206 may be used by individual 106 to engage in audio/video conferencing with healthcare provider 114, or with another trusted individual such as a family member. In other aspects, any kind of personal computing device (e.g., a tablet computer or a desktop computer, not depicted in FIG. 2) may be used by individual 106 for audio-visual communication with healthcare provider 114 or a trusted individual.

### Video Capturing and Data Processing

### Video Capturing:

In one aspect, there are two ways in which video and snapshots can be captured by cameras 208 and 210:
1) Upon Detecting Motion or Change:
   a) When motion or a change associated with individual 106 is detected, the cameras 208 and 210 initiate streaming of video and audio data.
   b) The streamed data is stored in, for example, an MP4 format (for video data) on a server on remote computing system 112 (not shown in FIG. 2). Video data may be stored in other formats associated with video data, such as AVI and MOV. The streamed data may also include audio data that may be stored in, for example, an MP3 format, or some other audio format, such as WAV and FLAC. The streamed data may include data from multiple sensing systems operating in different environments.
   c) Essential metadata, including timestamps, location coordinates, camera ID, and specific room or area being monitored, is included to enhance the contextuality of the data.
2) When There is No Activity for a Certain Period:
   a) If no motion or change is detected for a specified time, the cameras 208 and 210 take a snapshot for processing.
   b) This process ensures continuous monitoring by the AI caregiver system, even during periods of inactivity, to maintain safety.

The logic of capturing video based on motion or after a specific period of no motion significantly reduces the bandwidth required for processing, thereby optimizing efficiency.

### Data Processing:

1) Object and Action Recognition:
   a) In an aspect, a fine-tuned object and action recognition model (e.g., Model 2 238) processes the streaming video. In an aspect, model 2 238 runs locally on edge computing system 212 if there is sufficient computing power (not shown in FIG. 2). In an aspect, edge computing system 212 is integrated into one or both of cameras 208 and 210. In another aspect, cameras 208, 210 and edge computing system 212 may be integrated into a single unit (e.g., sensing system 104). If more computing power is needed than is available on edge computing system 212, then model 2 238 is deployed on remote computing system 112, as depicted in FIG. 2.

In an aspect, model 2 238 generates frame-level predictions, and can recognize various objects and human activities, such as:
i. Person-related actions: e.g., standing, lying down, sitting, and walking.
ii. Objects: e.g., beds, chairs, furniture, doors, bags, and wheelchairs.
   b) Model 2 238 contextualizes the scene by distinguishing between day and night modes and RGB and IR images. It counts the number of people present, adding depth to the analysis. Additionally, it classifies individuals as residents or caregivers.
   c) Along with textual information about the images and video, one or more raw embeddings are also stored by the AI Caregiver system. These embeddings enable the system to extract relevant features and then do a nearest neighbor search at an embeddings level.

In one aspect, an embedding is a compact summary or fingerprint of complex data, such as a video frame, image, or sentence, converted into a set of numbers that a computer (e.g., remote computing system 112) can understand. Instead of comparing raw data (like images pixel-by-pixel), remote computing system 112 compares these embeddings, which capture an essence or meaning of the content.

### *How Embeddings are Useful for Virtual Care (VCare) as Implemented by the AI Caregiver System:

In VCare, embeddings allow the AI Caregiver to recognize patterns such as falls, bed exits, or abnormal behavior by comparing the current situation to previously seen scenarios, quickly and efficiently. This is done through something called a nearest neighbor search, which finds the most similar past event based on these embeddings.

### *Storage of Embeddings:

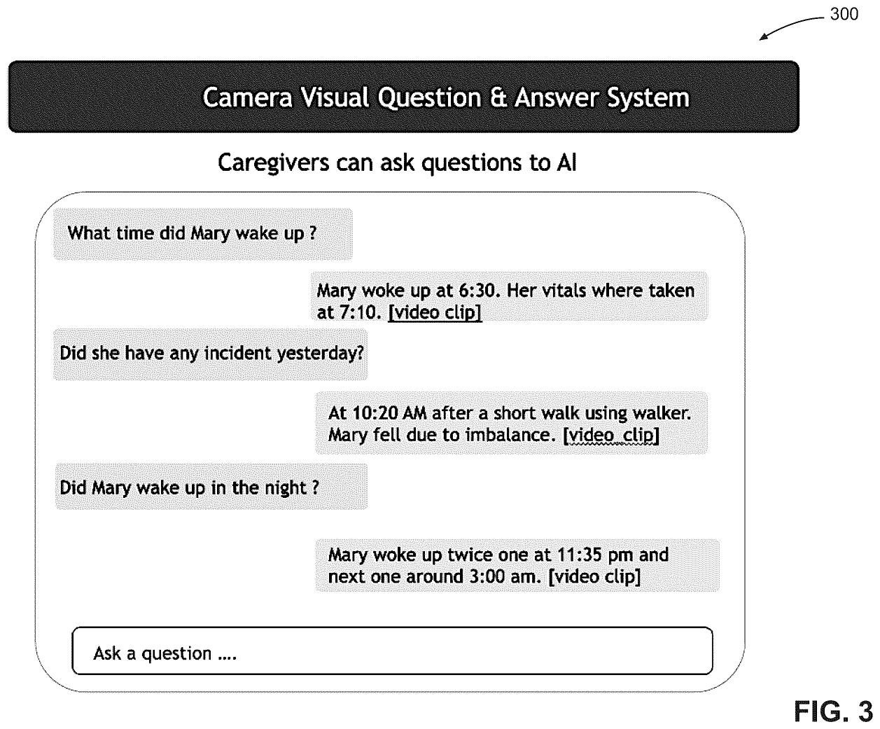

Embeddings are typically stored in a vector database or a dedicated embedding store on remote computing system 112, which allows fast searching and comparison. Common storage systems include services like Pinecone, Weaviate, FAISS, or even MongoDB with vector indexing. For VCare, this storage enables instant matching and decision-making across large video and event datasets.
2) Decision Engine and Model Interaction:
   a) In an aspect, decision engine 236 analyzes the results from the object/action recognition model to determine the need for a more advanced AI model (e.g., model 3 234). This decision depends on the monitoring task assigned to the camera(s) 208 and 210, and the characteristics and confidence level of the detected events.
   b) The decision engine 236 utilizes an interface for function calling, enabling the selection of the appropriate model and parameters for further analysis. This approach ensures that the processing is flexible and context-aware, catering to specific requirements such as detailed anomaly detection or complex behavior analysis.
3) User Settings and Configuration Management
   a) Some aspects of the AI caregiver system include one or more Natural Language Processing (NLP) components. In a traditional software interface, users have to select predefined options to configure certain settings. However, in the AI caregiver system, users (e.g., healthcare provider 114) can communicate their preferences to the AI caregiver system using natural language, such as voice or text (i.e., using NLP features included in the AI caregiver system). This interaction allows the AI caregiver to respond with additional options if it needs clarification, making it easy for users to set up one or more AI caregivers. For instance, if a user wants the AI Caregiver to monitor a resident between 10 AM and 2 PM, they can simply type this request in a chat option section. An example of such a text interaction is presented in FIG. 3.
   b) Some examples of alert configurations input by healthcare provider 114 to remote computing system 112 (i.e., the AI caregiver system) include:
   “I want you to alert me if the resident is walking more at night in rooms 101, 103, and 106. However, do not alert me if they wake up once or twice.”
   c) Another example of setting up a monitoring or dashboarding task is:
   “Please provide me with a daily aggregate of the number of times a resident gets up from bed and walks.”

Such alerts may be output by alert system 232 for viewing by healthcare provider 114.
4) Output Management and Data Utilization:
   a) The inputs to Model 2 238, including video/frames and their corresponding outputs, are vectorized and stored in a vector database for Question and Answer (Q&A) Retrieval Augmented Generation (RAG) (i.e., visual Q&A system 240).
   b) Additionally, the output of Model 2 238 is utilized to guide the training of Model 1 on edge computing system 212 through a continuous active learning process and teacher-student model.
   c) This iterative approach enhances the overall accuracy and responsiveness of the system over time.

The above description highlights the system's integrated approach, combining robust hardware with advanced AI-driven software, to provide a comprehensive monitoring solution tailored for senior care environments.

In the AI Caregiver System, the various AI models (e.g., model 1, model 2 238 and model 3 234) interact with the decision engine 236 to perform specific tasks. In an embodiment, the system leverages these three distinct AI models, each with a defined role, to ensure precise and context-aware responses to the dynamic environment of senior care facilities.

### AI Models and Their Functions

### Model 1: Change Detection in Audio/Video

Deployed directly on the camera(s) 208 and 210 and/or on edge computing system 212, model 1 initiates the system's response chain by detecting any motion within its field of view. This model is critical for identifying potential events that require further analysis, such as movement at unusual times or in specific areas that could indicate an emergency.

### Model 2 238: Object/Action Recognition

Running on remote computing system 112 (e.g., on the cloud), Model 2 238 processes the video clips sent from the cameras 208 and 210 after a change is detected. This model performs detailed object and action recognition, identifying key elements like persons, furniture, and doors, as well as specific actions like standing, sitting, or lying down. This model also categorizes video frames based on lighting conditions (day or night) and image type (RGB or IR), providing enriched metadata.

### Model 3 234: Generalized Vision AI

Model 3 234 is a more sophisticated multi-modal AI model, such as GPT-40 or Gemini, designed to perform complex visual and contextual analyses. This model processes the detailed inputs from Model 2 238 along with the specific task descriptions for the room it monitors, encoded in natural language. Model 3 234 analyzes the context and generates outputs structured according to predefined requirements tailored for each specific monitoring scenario.

### Decision Engine and Task Management 236

The decision engine 236 acts as the central coordinator within the system. The

instructions imputed by the caregiver (i.e., healthcare provider 114) via natural language is stored in a specific format. These instructions are used by the decision engine 236 every time it receives a task to validate some request. For example, in a room 102, the AI Caregiver may be tasked with: a) Detecting falls and alerting the on-shift human caregiver (i.e., healthcare provider 114) immediately. b) Warning the resident not to stand if they are sitting at the edge of the bed, while also alerting the caregiver (i.e., healthcare provider 114) about this potentially risky behavior. In an aspect, the decision engine 236 is located on remote computing system 112 (e.g., as a cloud-based platform), and has access to user configuration.

The decision engine 236 processes the prediction results from model 2 238 along with metadata information provided by model 2 238, evaluating these results against the caregiving tasks specified for each camera in different environments/rooms. The decision engine 236 then formulates a prompt that incorporates these data and the specific response required, directing model 3 234 to analyze the situation according to the set criteria.

Upon receiving the output from model 3 234, the decision engine 236 interprets these results to determine the appropriate action:

Alerting: If an urgent issue is detected, such as a fall, the AI caregiver system triggers an immediate alert to the caregivers.

Monitoring & Reporting: In less critical but noteworthy situations, the AI caregiver system may continue to monitor the resident more closely, adjusting its sensitivity to changes in activity or behavior.

Question & Answer Bot: The AI caregiver system can also initiate communication, either through audio warnings to the resident or alerts to the caregivers, based on the assessed needs and safety protocols.

This dynamic interaction between the AI models and the decision engine 236 ensures that each resident receives tailored/customized, attentive care, significantly enhancing both safety and quality of life in senior care facilities. The AI caregiver system's ability to adapt its responses based on real-time analysis and predefined guidelines exemplifies a significant advancement in automated caregiving technology.

In an aspect, the AI caregiver system is configured to monitor multiple individuals such as individual 106, in different environments (e.g., environment 102). The system may present corresponding status updates and/or alerts for each monitored individual to one or more healthcare providers such as healthcare provider 114.

### AI Caregiver System Functional Capabilities

The following is a list of tasks the AI caregiver system is able to perform, presented as a brief summary of each along with some context and examples.

### Alert:

1. Visual Monitoring for Safety: The AI caregiver system can continuously

monitor the resident (e.g., individual 106) for safety concerns, such as detecting falls, bed-exit(s) or unusual movements that might indicate distress or health issues. A top priority of the AI caregiver system is to ensure safety of the residents.
2. Audio Monitoring for Emergency Detection: The microphone can pick up sounds that indicate emergencies, like calls for help, or detect signs of discomfort or pain through vocalizations.

### Monitoring:

3. Communication Aid/Virtual Visits: The AI caregiver system can facilitate communication between the resident and caregivers or family members. Residents can voice their needs or concerns, and caregivers can respond through the speaker. Family members or an associated care team can broadcast messages which will be read out aloud for the residents in a voice that is familiar to them. Such a communication system is implemented via, for example, mobile device 204 and laptop computer 206, or any other personal computing device(s) used by individual 106.
4. Medication Reminders: The AI caregiver system can provide audible reminders for medication schedules, ensuring residents (e.g., individual 106) take their medications on time. The AI caregiver system may be configured to monitor the room environment (e.g., environment 102) to see if residents have in fact taken their medication. The input to the AI caregiver system is the video source from the camera (e.g., cameras 208 and 210).
5. Activity Monitoring: Monitoring the residents' activity levels and movement patterns to assess their mobility and alert caregivers if there is a significant decrease in activity.
6. Sleep Monitoring: Using the camera to observe sleep patterns and quality, alerting caregivers (e.g., healthcare provider 114) to potential sleep disturbances or irregular sleep behaviors.
7. Companionship and Engagement: Offering spoken word activities like reading books, playing music, or providing news and updates to keep residents engaged and mentally stimulated.
8. Voice-Activated Commands: Residents (e.g., individual 106) can use voice commands to control room features like lights or call for assistance, enhancing their sense of independence.
9. Instructional Assistance: Providing verbal instructions or reminders for daily tasks, such as eating times, hygiene routines, or exercise schedules. This is especially important for residents with dementia.
10. Mood and Well-being Checks: Using voice analysis to gauge the resident's mood and overall well-being, and alerting staff if there are signs of emotional distress or loneliness.
11. Environmental Monitoring: Alerting staff if the camera detects issues in the environment, such as a spill, fire, or other hazards.
12. Hygiene Monitoring: Checks when the resident's room is cleaned and sheets are changed per the code.
13. Loneliness and Depression Monitoring: The AI caregiver system virtually monitors the daily motion, other activities, sleep pattern and facial expressions of individual 106 to determine an associated loneliness index and depression score.
14. Daily Activity Timeline: The AI caregiver system records all the important activity happening each day in every room along with visual snapshots and also a link to the video. This can be helpful during the healthcare providers' shift change and handovers.

### AI Models and Algorithms

1. Change Detection (motion+audio)
   *Algorithm: Model 1 on edge computing system 212 utilizes a background subtraction algorithm to detect a change in the motion within the camera's field of view. This method involves comparing successive video frames to identify significant pixel differences, indicating movement. This approach is effective in distinguishing between relevant motion events and static backgrounds, thereby optimizing the processing load by focusing only on frames with detected motion. For audio based change detection, a spectral-based change detection method is used to detect change in the audio.
   *Parameters Adjustment: The sensitivity and parameters of the motion and audio detection algorithm are dynamically adjusted based on environmental conditions, such as lighting changes and typical activity patterns in the monitored area (e.g., environment 102). This adaptability enhances the accuracy and efficiency of the motion and sound detection process.
2. Vision Transformer
   *Processing Method: Once Model 1 triggers a capture, a video clip is forwarded to Model 2 238 for deeper analysis. In one aspect, the video clip is 30 seconds in length. In other embodiments, the video clip can be of an arbitrarily long temporal length. Operating on remote computing system 112 (e.g., in the cloud), Model 2 238 employs advanced object detection and action recognition techniques to analyze each frame individually. This model identifies and classifies various objects and actions within the video, providing detailed metadata about the scene. Lightweight vision transformer based state-of-the-art models are some of the methods that can be used for this process.
   *Functionality: Model 2 238 is capable of detecting a wide range of entities and actions, such as people in various postures (standing, sitting, lying down), furniture, doors, and more. This model also classifies frames based on visual cues, distinguishing between day and night conditions and between RGB and IR imaging, which is crucial for accurate context interpretation in varying lighting conditions. Model 2 238 acts as a lightweight video context analysis model. Model 2 238 extracts objects from each frame along with human context and orientation. This detailed frame-level annotation helps the model 3 234 to decide the type of action it needs to perform.
3. Decision Engine 236
   *Processing Method: Input to decision engine 236 is a video (e.g., 30 seconds in length) along with the object and action recognition results including the metadata information for each frame of the video. The other inputs to the model (i.e., decision agent 236) are user choices and preferences (e.g., user settings 214) which are stored in the database 222, and pulled appropriately for each call. Using these two inputs, the decision agents (e.g., decision engine 236) then decide on which of the actions function needs to be called and the suitable prompt from the associated prompt library. For instance if the user's preference (e.g., user preferences/settings 214 for healthcare provider 114) is to detect a person laying down on the floor and alert human caregivers, then the agent (e.g., decision engine 236) decides to pass the video along with its metadata and predefined prompt to detect a fall to the next Model 3 234.
   *Functionality: The decision agent (e.g., decision engine 236) acts as an intelligent routing block that combines input from the video data and user configuration and sends the request to model 4 (a vision language model similar in functionality to model 3 234 but not depicted in FIG. 2) with appropriate prompts.
4. Vision Language Model
   *Processing Method: The input to the vision language model may be a custom prompt based on the user preference and the task in hand along with the videos and its frame level detection data. either In one aspect, a multi-modal model such as GPT-4o or Gemini 1.5 model may be used to implement the vision language model. The prompt also contains information on what the output format should look like. With these inputs, one or more vision language models process the request and output the data based on a predefined format. This output is again sent back to the decision agent, which then decides to perform another set of actions such as notifying the customers, storing the received information in the database for weekly analytics etc.
   *Functionality: Model 4 acts as a generalized video analysis agent that can perform tasks that are requested via a prompt. It also acts as a teacher to teach the student Model 2 238. The output of Model 3 234 is stored, and when the model 2 238 degrades, the performance based on results from model 3 234 is used to improve an output accuracy of model 2 238.

FIG. 3 is a screenshot 300 depicting an interaction between a human caregiver and an automated caregiver system. In an aspect, healthcare provider 114 interacts with remote computing system 112 via a graphical user interface that includes a chat-based interactive interface such as the interface shown in screenshot 300. Computing system 112 may include one or more NLP-based algorithms that implement such interactions. Screenshot 300 depicts an interactive chat session between healthcare provider 114 and the AI caregiver system.

### Visual Q&A bot:

*Interactive AI Caregiver:

The healthcare provider 114 can interact with the AI Caregiver by asking questions about the conditions in room 101. The AI caregiver responds with detailed answers or a summary of the situation, accompanied by visual examples (e.g., video clips and/or still images captured by one or more cameras included in sensing system 104). This interactive communication allows the healthcare provider 114 to ask various questions, and the AI caregiver will provide corresponding responses and video clips as references. This feature helps prevent hallucinations and offers a convenient way for human caregivers to validate the AI caregiver's responses.

As depicted in screenshot 300, a caregiver (i.e., healthcare provider 114) asks the AI caregiver system questions about a resident, Mary. The AI caregiver system responds with appropriate responses that better inform the caregiver about how the individual (e.g., individual 106) is doing.

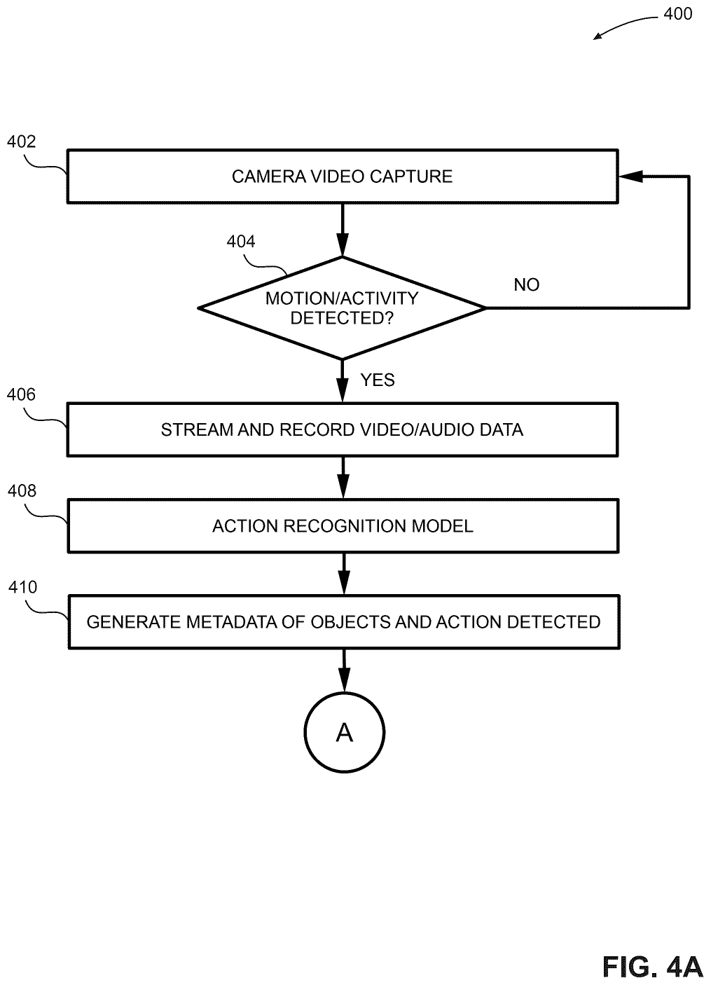

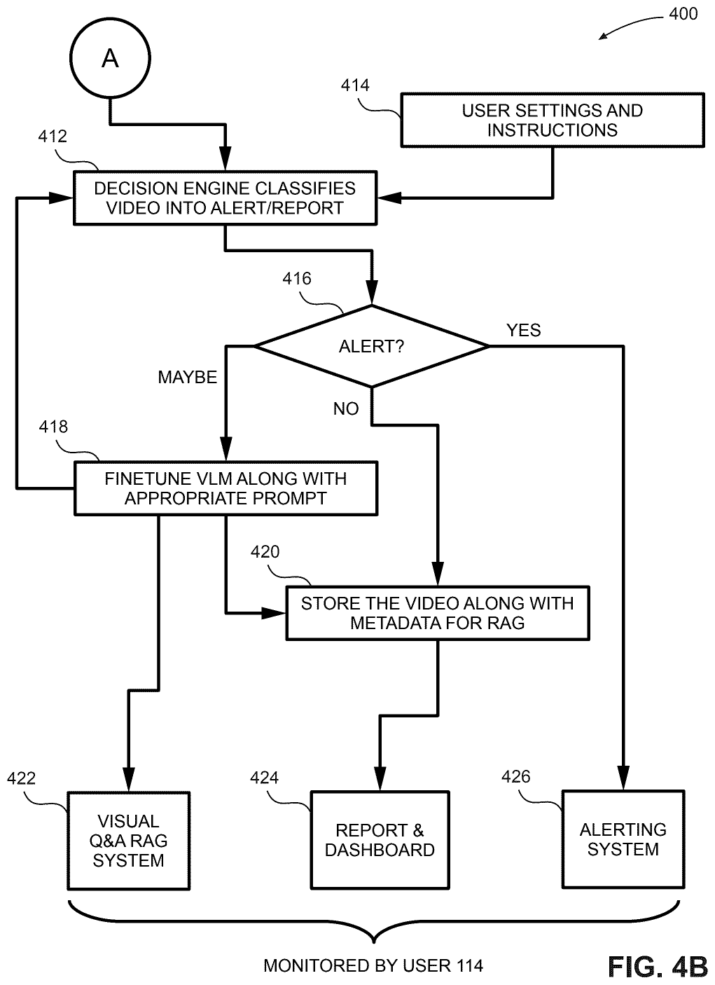

FIG. 4A is a flow diagram depicting a method 400 for automated caregiving.

Method 400 may include a camera video capture (402). For example, one or more cameras included in sensing system 104 (e.g., cameras 208 and 210) may capture video of individual 106. Method 400 may include determining whether motion and/or activity is detected with respect to individual 106 (404). For example, this task may be performed by edge computing system 212. If no motion and/or activity is detected, then the method goes back to 402.

If motion and/or activity is detected at 404, then method 400 may include streaming and recording video/audio data (406). For example, sensing system 104 that may include cameras 208 and 210, and edge computing system may record video and stream the video data to remote computing system 112.

The video stream data may be processed by an action recognition model (408). In an aspect, model 2 238 may perform the tasks related to action recognition. Method 400 may include the action recognition model generating metadata of objects and actions (410). For example, model 2 238 may generate metadata of objects in environment 102, and one or more actions associated with individual 106 (e.g., eating, sleeping, walking, falling, etc.).

Moving on to FIG. 4B, method 400 includes a decision engine classifying video data into an alert or a report (412). At 412, a decision engine (e.g., decision engine 236) receives the video data generated at 406, and metadata at 410, along with user settings and instructions 414. In an aspect, user settings and instructions 414 may be similar to user settings 214. At 412, decision engine 236 may also receive outputs from a visual language model (VLM). Based on the information received, the decision engine 236 may determine whether to generate an alert (416).

If, at 416, decision engine 236 determines that an alert needs to be generated, then method 400 generates an alert via an alerting system (426). For example, remote computing system 112 may generate an alert for review by healthcare provider 114 via alert system 232.

If, at 416, decision engine 236 determines that an alert does not need to be generated, then method 400 stores the video along with the metadata for RAG (420). Method 400 may then generate a report and present the report to healthcare provider 114 via a dashboard (424). In an aspect, the dashboard is presented to healthcare provider 114 via a graphical user interface.

If, at 416, decision engine 236 is not certain about whether an alert needs to be generated, then method 400 goes to 418, where the VLM is fine-tuned along with an appropriate prompt. The fine-tuning process 418 provides feedback for 412. Outputs from the fine-tuning process 418 are also stored at 420. From 418, method 400 presents a visual Q&A RAG system to healthcare provider 114 (422).

Method 400 may further include the following integration and data flow:
1. Data Capture and Initial Processing (402-406)
   *The integrated camera(s) (e.g., cameras 208 and 210) is/are equipped with sensors that continuously monitor the environment. Upon detecting motion via model 1, the system captures a 30-second video clip, which is immediately encrypted and transmitted to the cloud (e.g., remote computing system 112) for further analysis by model 2 238.
   *This process ensures that only relevant data-video clips where motion is detected—are sent for further processing, significantly reducing unnecessary data transmission and storage.
2. Cloud Processing and Decision Making (408-410)
   *In the cloud (e.g., on remote computing system 112), model 2 238 processes the received video to extract actionable insights and preliminary assessments. This data, along with the extracted metadata, forms the basis for the next level of analysis.
   *The processed data and insights from model 2 238 are then passed to the decision engine 236, which evaluates the information against predefined criteria specific to each monitored room. This evaluation determines whether further analysis by model 3 234 is necessary.
3. Advanced Analysis and Response Activation (412-420)
   *If the decision engine deems that further analysis by model 3 234 necessary, detailed prompts based on outputs from model 2 238 and the specific operational requirements of the camera's location are formulated and sent to model 3 234. Model 3 234 then conducts a comprehensive analysis to generate a structured response tailored to the situation.
   *The decision engine receives outputs from model 3 234, interprets the results, and implements the appropriate actions, whether triggering an alert, continuing monitoring, or initiating communication through the system's two-way audio capabilities.
4. Function Calling for each set of activity (422-426)
   *For each activity, a dedicated function is implemented in the backend. For instance if the user wants to analyze the activities of daily living (ADL) of a resident, then there is an ADL monitoring function which performs one or more predefined steps.
   *This technical specification outlines the interplay between hardware and software components, demonstrating the AI caregiver system's capability to provide timely and accurate responses through advanced AI-driven analysis and decision-making processes.

### Implementation Scenario

### Assisted Living, Memory Care and Hospitals

Assisted living and memory care facilities provide housing, personal care, and

supportive services to older adults who need assistance with activities of daily living (ADLs) and may have cognitive impairments. The implementation of AI caregiver system in assisted living and memory care settings can enhance the quality of life for residents and improve the efficiency and effectiveness of care delivery.

### Key Considerations:

*Resident Needs: Residents in assisted living and memory care facilities have varying needs and abilities. Some may require assistance with basic ADLs, such as bathing, dressing, and eating, while others may have more complex needs, such as managing medications or cognitive stimulation.
*Staffing: Assisted living and memory care facilities typically have a high staff-to-resident ratio to ensure that residents receive the care they need. However, staff turnover can be high, and it can be challenging to find and retain qualified caregivers.
*Regulatory Compliance: Assisted living and memory care facilities must comply with various federal, state, and local regulations. These regulations include requirements for staff training, infection control, and resident safety.

### Implementation Plan:

*Assessment and Planning: Conduct a thorough assessment of the needs of the assisted living and memory care facility, including the number of residents, staff, and the types of services provided.
*Staff Training: Provide comprehensive training to staff on how to use the AI caregiver system effectively. This training should cover topics such as system setup, operation, and troubleshooting.
Resident Education: Educate residents and their families about the AI caregiver system. Explain how the system can help residents stay safe, independent, and connected.
*System Installation: Install the AI caregiver system according to the manufacturer's instructions. This may involve mounting devices in resident rooms, setting up wireless networks, and configuring the system software.
*System Monitoring: Regularly monitor the AI caregiver system to ensure that it is functioning properly. This may involve checking the system's logs, reviewing resident activity data, and conducting periodic system audits.
*Evaluation: Evaluate the effectiveness of the AI caregiver system on a regular basis. This may involve collecting feedback from residents, staff, and families, and reviewing data on resident safety, independence, and social engagement.

### Benefits of Implementation:

*Improved Resident Safety: The AI caregiver system can help to improve resident safety by providing real-time monitoring of residents' activities and alerting staff to potential emergencies.
*Increased Resident Independence: The AI caregiver system can help residents maintain their independence by allowing them to control their environment and summon/request assistance when needed.
*Enhanced Social Engagement: The AI caregiver system can help residents stay connected with their loved ones and participate in social activities.
*Reduced Staff Burden: The AI caregiver system can help to reduce the burden on staff by automating tasks and providing real-time information about residents' needs.
*Improved Compliance: The AI caregiver system can help assisted living and memory care facilities to comply with regulations by providing documentation of resident care and safety.
   Features of the AI caregiver system
*The AI caregiver system is equipped with the ability to passively observe the room around the clock, 24 hours a day, 7 days a week. This constant monitoring allows the AI caregiver system to continuously generate detailed summaries of what is observed, including visual and auditory information, related to individual 106.
*Users (e.g., healthcare provider 114) can prompt the AI caregiver system on what it needs to do, and assign duties accordingly. In an aspect, interactions between a human caregiver (e.g., healthcare provider 114) and the AI caregiver system are done via NLP methods. These duties are entered in a textual format e.g., “Please alert me if there is a fall or if the resident is trying to exit the bed”.
*The insight generated from the AI caregiver system is stored in a format that caregivers can ask questions and get responses from this information.
*The AI caregiver system can also run periodic predefined queries around these texts and the system will be able to provide answers along with the snapshot. This is the monitoring and report generation feature.
*The AI caregiver system can monitor sleep patterns and generate sleep scores.
*Healthcare providers (e.g., healthcare provider 114) can monitor the activities of daily living and classify each set of motions/actions into one of the activities. Caregivers (e.g., healthcare provider 114) can then analyze each activity over a period of time and see how the progress of the residents and provide their observation. E.g., their walking ability is improving, constant or degrading.
*Monitor fall and bed-exit data, with four AI-based models helping the system to identify incidents.
*Ability for human caregivers (e.g., healthcare provider 114) or family to communicate with the AI caregiver system using easy to use Natural Language and instruct it to do specific things. A functionality called voice direct message (DM) can be implemented, where a caregiver (e.g., healthcare provider 114) can have a predefined voice stored and send the voice samples over to the camera.
*Residents (e.g., individual 106) can ask the AI caregiver system to help find something that they have kept somewhere using voice based interaction.
*The AI caregiver system can initiate a natural human-like conversation with the residents (e.g., individual 106) when requested or per the configuration.
*Function calling for each set of activities. Each set of activities is defined as a function that has a set of steps. When a user (e.g., healthcare provider 114) wants certain queries to be executed the AI caregiver system will be able to execute whichever function it needs to call. E.g., if the user wants to analyze ADL for room 102 then an ADL function with a UUID of camera in room 102 is passed. The function returns a result in a certain format.

In general, the AI Caregiver System with its integrated camera, advanced AI models, and responsive decision engine, offers a transformative approach to care both in senior living and hospitals. By leveraging generative AI, deep learning and natural language processing, the system can analyze video and audio data in real time, accurately detecting and responding to resident needs and emergency situations.

The collaboration of three distinct AI models-Model 1 for motion detection, Model 2 238 for object and action recognition, and Model 3 234 for generalized multi modal vision and audio-empowers the system to provide tailored care for each resident. The decision engine 236 acts as the central coordinator, directing the AI models to analyze data and take appropriate actions, such as triggering alerts, monitoring activity, and facilitating communication.

This system significantly enhances the efficiency of caregivers, allowing human caregivers to focus on providing personalized and compassionate care to residents. It also empowers residents by offering assistance with daily tasks, medication reminders, and companionship, promoting their independence and quality of life.

As AI technology continues to advance, the Virtual AI Caregiver System has the potential to revolutionize the way senior care is delivered. By leveraging real-time monitoring, intelligent decision-making, and proactive caregiving, this system can make a meaningful difference in the lives of seniors and their loved ones, ultimately leading to a safer, more fulfilling, and connected living experience.

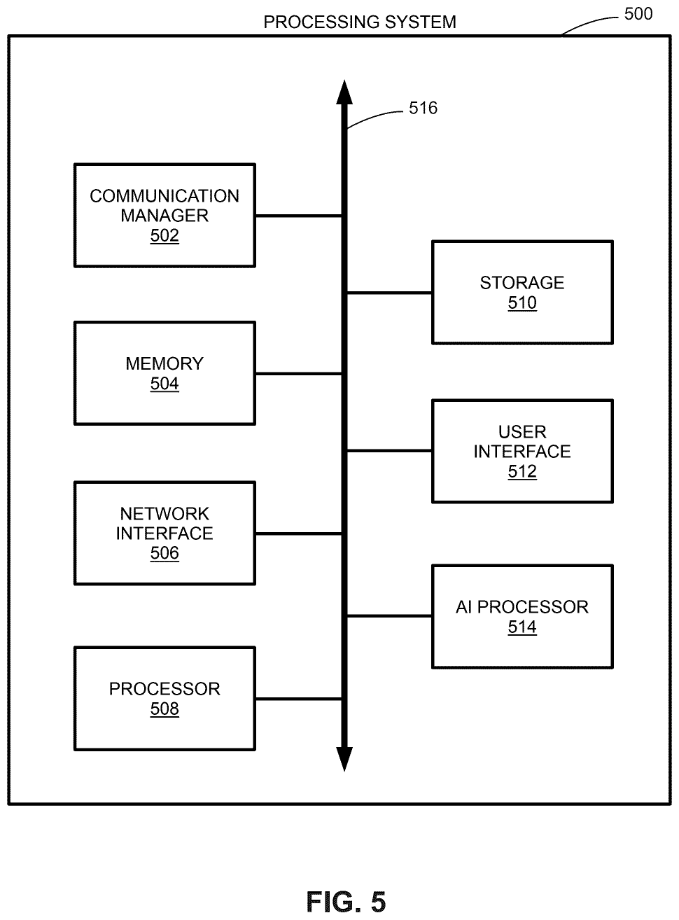

FIG. 5 is a block diagram of a computing system 500. As depicted, processing system 500 includes processing system architecture includes communication manager 502, memory 504, network interface 506, processor 508, storage 510, user interface 512, AI processor 514, and system bus 516.

Processing system 500 may be used to implement aspects of the systems and methods described herein. For example, processing system 500 can be used as a basis for implementing aspects of remote computing system 112 and/or edge computing system 108.

In an aspect, communication manager 502 is configured to manage communication protocols and associated communication with external peripheral devices as well as communication with other components in remote computing system 112 and/or edge computing system 108.

In an aspect, memory 504 includes a non-transitory computer medium. Memory 504 may be comprised of any combination of volatile and non-volatile memory components. Examples of components that may be used to implement memory 504 include random-access memory (RAM), read-only memory (ROM), electrically-erasable programmable read-only memory (EEPROM), flash memory, magnetic memory, optical memory, and so on. Memory 504 may include machine-readable instructions that may be executable by a processor such as processor 508. These machine-readable instructions when executed by the processor 508 cause the processor 508 to perform one or more method steps of an embodiment described herein.

Network interface 506 may be used to interface processing system 500 with other computing devices and/or computer networks. Examples of computer networks include a local area network (LAN), a wide area network (WAN), the Internet, and so on. Network interface 506 may support any combination of wired and wireless connectivity/communication protocols such as Ethernet, Wi-Fi, Bluetooth, ZigBee, etc.

A processor 508 included in some embodiments of processing system 500 is configured to perform functions that may include generalized processing functions, arithmetic functions, and so on. Processor 508 is configured to process information associated with the systems and methods described herein. Processor 508 may be configured as any combination of microcontrollers, microprocessors, digital signal processors (DSPs), field-programmable gate arrays (FPGAs), graphics processing units (GPUs), accelerated processing units (APUs), central processing units (CPUs), neural processing units (NPUs), application-specific integrated circuits (ASICs), and so on. Processor 508 may be embodied as a single-core processor, or a multi-core processor. Processor 508 may be implemented as a centralized processor, or in a distributed manner (e.g., a distributed computing system).

Processing system 500 may include storage 510, that further includes one or more long-term storage devices such as hard disk drives, magnetic drives, magnetic tape, optical storage media (e.g., compact disks (CDs) or digital versatile disks (DVDs)), and so on. Storage 510 may be implemented as a non-transitory computer-readable medium. Storage 510 may be configured to store data and/or instructions related to the operation of processing system 500.

User interface 512 allows other devices or a user to interact with embodiments of the systems described herein. User interface 512 may include any combination of user interface devices such as a keyboard, a mouse, a trackball, one or more visual display monitors, touch screens, incandescent lamps, LED lamps, audio speakers, buzzers, microphones, push buttons, toggle switches, and so on. User interface 512 may alco include interfaces such as USB, Thunderbolt and Fire Wire.

AI processor 514 may be configured to implement one or more AI-related components that implement the workflows and processes of the systems and methods described herein.

System bus 516 communicatively couples the different components of processing system 500, and allows data and communication messages to be exchanged between these different components.

Processing system 500 may be used to implement aspects of remote computing system 112 and/or edge computing system 108.

Although the present disclosure is described in terms of certain example embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the scope of the present disclosure.

What is claimed is:

1. A system comprising:
a sensing system configured to monitor an individual, the sensing system including at least one camera and at least one microphone, wherein the at least one camera is configured to capture any combination of video and images of an individual being monitored and at the least one microphone is configured to capture audio signals generated by the individual;
an edge computing system connected to the sensing system and configured to receive one or more sensed signals associated with the sensing system monitoring the individual, wherein the sensed signals include the video or images, and the audio signals; and
a remote computing system connected to the edge computing system via a network, the remote computing system configured to:
receive the sensed signals from the edge computing system;
process the sensed signals using at least one artificial intelligence (AI)-based system; and
provide assistance to the individual being monitored based on the processing.

2. The system of claim 1, wherein the assistance provided is any combination of:
providing reminders to assist the individual with carrying out their daily activities of life; and
generating an alert to a human caregiver in case of an emergency associated with the individual.

3. The system of claim 1, further comprising a two-way communication channel, wherein a human caregiver or a family member associated with the individual can interact with the individual using the communication channel.

4. The system of claim 3, wherein the two-way communication channel is any combination of a voice communication channel, a video communication channel, and a text communication channel.

5. The system of claim 1, wherein the processing includes fall monitoring associated with the individual.

6. The system of claim 1, wherein the processing includes monitoring the individual's sleep patterns.

7. The system of claim 6, wherein the processing includes generating one or more sleep scores based on the monitoring.

8. The system of claim 1, wherein a human caregiver can interact with the AI-based system and configure the AI-based system with one or more required tasks or request one or more updates associated with monitoring the individual, wherein the interaction is implemented using natural language processing (NLP).

9. The system of claim 1, further comprising generating an alert if an emergency is detected responsive to the processing.

10. The system of claim 9, wherein a human caregiver or the AI-based system can interact with the individual to address the emergency.

11. A method comprising:
monitoring, by a sensing system, an individual, wherein the sensing system includes least one camera and at least one microphone, wherein the at least one camera is configured to capture any combination of video and images of an individual being monitored and at the least one microphone is configured to capture audio signals generated by the individual as a part of the monitoring;
receiving, by an edge computing system connected to the sensing system, one or more sensed signals associated with the sensing system monitoring the individual, wherein the sensed signals include the video or images, and the audio signals;
receiving, by a remote computing system connected to the edge computing system via a network, the sensed signals from the edge computing system;
the remote computing system processing the sensed signals using at least one artificial intelligence (AI)-based system; and
the remote computing system providing assistance to the individual being monitored based on the processing.

12. The method of claim 11, wherein the assistance provided is any combination of:
providing reminders to assist the individual with carrying out their daily activities of life; and
generating an alert to a human caregiver in case of an emergency associated with the individual.

13. The method of claim 11, further comprising a two-way communication channel, wherein a human caregiver or a family member associated with the individual can interact with the individual using the communication channel.

14. The method of claim 13, wherein the two-way communication channel is any combination of a voice communication channel, a video communication channel, and a text communication channel.

15. The method of claim 11, further comprising fall monitoring associated with the individual.

16. The method of claim 11, further comprising monitoring the individual's sleep patterns.

17. The method of claim 16, further comprising generating one or more sleep scores based on the monitoring.

18. The method of claim 11, wherein a human caregiver can interact with the AI-based system and configure the AI-based system with one or more required tasks or request one or more updates associated with monitoring the individual, wherein the interaction is implemented using natural language processing (NLP).

19. The method of claim 11, further comprising generating an alert if an emergency is detected responsive to the processing.

20. The method of claim 19, wherein a human caregiver or the AI-based system can interact with the individual to address the emergency.

21. The system of claim 1, wherein the edge computing system includes at least one speaker to send one or more audio signals to the individual.
